(19) United States
(12) Patent Application Publication
(10) Pub. No.: US 2022/0189582 A1
(43) Pub. Date: Jun. 16, 2022

(54) ANTIBODY LIBRARY CONSTRUCTION METHOD AND DEVICE BASED ON DEEP LEARNING

(71) Applicant: CUSABIO TECHNOLOGY LLC, Wuhan (CN)

(72) Inventors: Quangao HUA, Wuhan (CN); Qin SHU, Wuhan (CN)

(21) Appl. No.: 17/353,879

(22) Filed: Jun. 22, 2021

(30) Foreign Application Priority Data
Dec. 15, 2020 (CN) 2020114776827

(51) Int. Cl. G16B 35/10; G16B 40/00

(52) U.S. Cl. CPC G16B 35/10; G16B 40/00

(57) ABSTRACT

An antibody library construction method based on deep learning, comprising the steps of: obtaining a corresponding relation among antigen epitopes, antigen recognition regions and coding genes, and constructing a first database matching with the antigen epitopes, the antigen recognition regions and the coding genes; processing the antigen epitopes; carrying out clustering and characteristic extraction on the first database; and taking the multi-dimensional vector as the input of a temporal convolutional neural network, and stopping training until the error is lower than the threshold and tends to be stable to obtain the trained neural network model; and screening out antibody sequences having different activities, stability and specificity to the antigens in the coding gene sequence set X according to molecular docking, molecular dynamics and an existing gene sequence database Y so as to establish a secondary antibody library.

### CROSS-REFERENCE TO RELATED APPLICATIONS

The application claims priority to Chinese patent application No. 202011477682.7, filed on Dec. 15, 2020, the entire contents of which are incorporated herein by reference.

### TECHNICAL FIELD

The present disclosure relates to the field of biological information and deep learning, in particular to an antibody library construction method and device based on deep learning.

### BACKGROUND

An antigenic epitope is also known as an antigenic determinant (AD) and is a special chemical group determining the specificity of an antigen in an antigen molecule. The antigen binds to an antigen receptor on the surface of the corresponding lymphocyte through the antigenic epitope, thereby activating the lymphocyte and causing an immune response; and the antigen also specifically binds to a corresponding antibody or sensitized lymphocyte through the epitope to exert an immune effect.

A full set of antibody variable region genes are cloned through a DNA recombination technology; functional antibody molecule fragments are expressed in a prokaryotic system; and this full set of antibody gene expression library becomes an antibody library.

A phage is a DNA single-stranded virus ubiquitous in nature with a length of about 7000 bp. The phage genome codes eleven proteins. The phage display technology selects the first structural domain and signal sequence of the P protein to insert a foreign protein coding sequence. After foreign proteins are packaged and processed by the phage, the foreign proteins are expressed on the surfaces of virus particles. A phage M13-based phage display library can use a phagemid or phage vector system to code antibody-coat protein fusions, and multiple antibody fragments are displayed on the pIII minor coat protein. The phage antibody library technology is a technology for preparing new antibodies, and the autophage display technology is developed.

Parmley et al. described the phage surface expression technology for the first time in 1988. The antibody molecule is the first protein molecule that has a natural protein function and can be expressed on the surface of the phage. Due to the development and perfection of the phage vector system, the research and application of the phage antibody technology expand continuously, which has attracted widespread attention. Nucleotide sequences are randomly introduced in the CDR region, so that a more diverse phage antibody library can be artificially synthesized; and in order to obtain specific antibodies with high affinity, after positive clones are obtained, the CDR region of the specific antibody gene can be subjected to gene mutation screening. The emergence of the phage display technology and the continuous improvement of phage antibody expression and screening systems have realized that a variety of specific antibodies can be directly obtained without antigen immunization.

### SUMMARY

In order to reduce the problems of cumbersome screening, repeated adsorption, elution, and amplification in the traditional antibody library construction process, the present disclosure provides an antibody library construction method based on deep learning.

An antibody library construction method based on deep learning according to the present disclosure, comprising the following steps of:

obtaining a corresponding relation among antigen epitopes, antigen recognition regions and coding genes, and constructing a first database matching with the antigen epitopes, the antigen recognition regions and the coding genes;

processing the antigen epitopes by using a trained neural network model so as to obtain the coding gene sequence set X of antibodies to be predicted, wherein the trained neural network model is obtained by training according to the method which comprises the steps of: carrying out clustering and characteristic extraction on the first database sequentially according to a classification of antigens, a homology of amino acid residues in the antigen epitopes, and positions of the antigen recognition areas to obtain a multi-dimensional vector for predicting antibody genes; and taking the multi-dimensional vector as the input of a temporal convolutional neural network, and stopping training until the error is lower than the threshold and tends to be stable to obtain the trained neural network model; and

screening out antibody sequences having different activities, stability and specificity to the antigens in the coding gene sequence set X according to molecular docking, molecular dynamics and an existing gene sequence database Y so as to establish a secondary antibody library.

According to the present disclosure, the temporal convolutional neural network comprises at least two convolutional hidden layers and at least one residual error module, the output of at least one convolutional hidden layer is determined by a set number of latest label data, and the output of one convolutional hidden layer is determined by all label data, wherein the residual error module uses a Zero-padding method to ensure that dimensions of input data and output data are consistent.

According to the present disclosure, the screening out antibody sequences with different activities, stability and specificity to the antigens in the coding gene sequence set X according to molecular docking, molecular dynamics and the existing gene sequence database Y so as to establish the secondary antibody library comprises the steps of:

matching the coding gene sequence set X with the existing gene sequence database Y, calculating the similarity Si between coding gene sequences xi and existing gene sequences yᵢ, and arranging yᵢ in descending order of similarity;

taking the top 10 gene sequences of similarity as a candidate antibody sequence set, and establishing the secondary antibody library according to the activities, stability, and specificity of expression products of the candidate antibody sequence set.

According to the present disclosure, if the maximum value of the similarity Si of the candidate antibody sequences is lower than the threshold, the expression products of the candidate antibody sequences are subjected to molecular dynamics simulation or molecular docking with the antibodies in a simulated environment, and the activities, stability and specificity of the expression products are evaluated by a scoring function to establish the secondary antibody library.

According to another aspect of the present disclosure, an antibody library construction device based on deep learning, comprising a construction module, a model training module, and a screening module, wherein

the construction module is used for obtaining the corresponding relation among the antigen epitopes, the antigen recognition regions and the coding genes, and constructing the first database matching with the antigen epitopes, the antigen recognition regions and the coding genes;

the model training module is used for processing the antigen epitopes by using the trained neural network model so as to obtain the coding gene sequence set X of the antibodies to be predicted; the trained neural network model is obtained by training according to the a method which comprises the steps of: carrying out clustering and characteristic extraction on the first database sequentially according to the classification of the antigens, the homology of the amino acid residues in the antigen epitopes, and the positions of the antigen recognition areas to obtain the multi-dimensional vector for predicting the antibody genes; and taking the multi-dimensional vector as the input of the temporal convolutional neural network, and stopping training until the error is lower than the threshold and tends to be stable to obtain the trained neural network model; and

the screening module is used for screening out the antibody sequences having different activities, stability and specificity to the antigens in the coding gene sequence set X according to molecular docking, molecular dynamics and the existing gene sequence database Y so as to establish the secondary antibody library.

The screening module comprises a calculating module, a first screening module and a second screening module, wherein

the calculating module is used for matching the coding gene sequence set X with the existing gene sequence database Y and calculating the similarity Si between the coding gene sequences xi and the existing gene sequences yᵢ;

the first screening module is used for arranging yᵢ in descending order of similarity, taking the top 10 gene sequences of similarity as the candidate antibody sequence set, and establishing the secondary antibody library according to the activities, stability, and specificity of the expression products of the candidate antibody sequence set; and

the second screening module is used for molecular dynamics simulation or molecular docking of the expression products of the candidate antibody sequences and the antibodies in a simulated environment, and evaluating the activities, stability and specificity of the expression products by the scoring function to establish the secondary antibody library.

Another aspect of the disclosure includes an electronic device, comprising at least one processor and a storage device storing at least one executable program instruction, when executed by the at least one processor, cause the at least one processor to perform the antibody library construction method based on deep learning.

Another aspect of the disclosure includes a non-transitory computer-readable storage medium storing executable program instructions that, when executed by a processor, cause the processor to perform the antibody library construction method based on deep learning.

The antibody library construction method based on deep learning has the following beneficial effects:

1. candidate antibody genes are subjected to preliminary screening through the temporal convolutional neural network, so that on the one hand, the processes of screening, repeated adsorption, elution, and amplification in the conventional genetic engineering are reduced, on the other hand, the number of layers and calculated amount of a neural network model are reduced, and the ability of parallel processing of the neural network model is improved; and the antibody genes are further screened through molecular dynamics or molecular docking methods, so that the interpretability and accuracy of the model is improved; and

2. the temporal convolutional network supports variable input lengths and adapts to the data attributes of the gene sequence, so that the model has better generalization ability than the existing neural network model used for antibody library construction.

### BRIEF DESCRIPTION OF DRAWINGS

FIG. 1 shows a basic flowchart of an antibody library construction method based on deep learning in some embodiments of the present disclosure;

FIG. 2 shows a schematic structural diagram of a temporal convolutional neural network model in some embodiments of the present disclosure;

FIG. 3 shows a basic structure diagram of an antibody library construction device based on deep learning in some embodiments of the present disclosure;

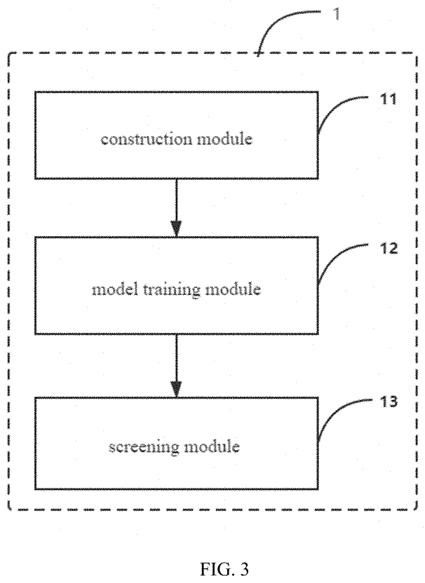

FIG. 4 shows a schematic structural diagram of a screening module in some embodiments of the present disclosure; and

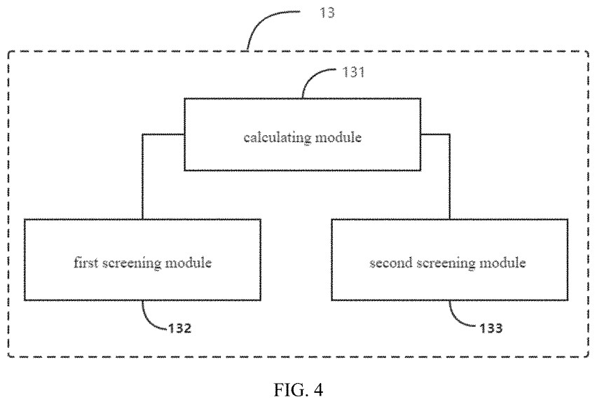

FIG. 5 shows a basic structure diagram of electronic equipment of the present disclosure.

### DETAILED DESCRIPTION OF THE EMBODIMENTS

The principles and characteristics of the present disclosure are described below with reference to the accompanying drawings. The examples given are only used to explain the present disclosure, not to limit the scope of the present disclosure.

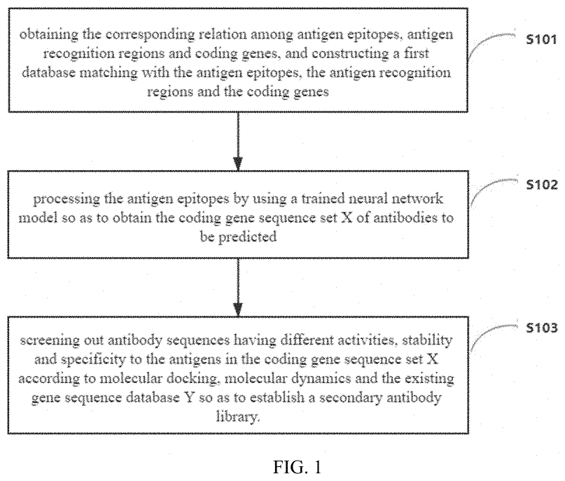

Referring to FIG. 1, the first aspect of the present disclosure provides an antibody library construction method based on deep learning. The antibody library construction method based on deep learning comprises the steps of: obtaining the corresponding relation among antigen epitopes, antigen recognition regions and coding genes, and constructing a first database matching with the antigen epitopes, the antigen recognition regions, and the coding genes; processing the antigen epitopes by using a trained neural network model so as to obtain the coding gene sequence set X of the antibodies to be predicted; and screening out antibody sequences having different activities, stability and specificity to the antigens in the coding gene sequence set X according to molecular docking, molecular dynamics and an existing gene sequence database Y so as to establish a secondary antibody library. Then the secondary antibody library is conventionally and genetically engineered to obtain antibody libraries with different activities, stability and specificity.

Exemplarily, when the antigen epitopes are amino acid residues, the corresponding antibody epitope coding refers to table 1:

TABLE 1
Amino acid coding comparison table

| | | Three-letter | |
|---|---|---|---|
| Chinese name | English name | abbreviation | One-letter symbol |
| [symbol] | Glycine | Gly | G |
| [symbol] | Alanine | Ala | A |
| [symbol] | Valine | Val | V |
| [symbol] | Leucine | Leu | L |
| [symbol] | Isoleucine | Ile | I |
| [symbol] | Proline | Pro | P |
| [symbol] | Phenylalanine | Phe | F |
| [symbol] | Tyrosine | Tyr | Y |
| [symbol] | Tryptophan | Trp | W |
| [symbol] | Serine | Ser | S |
| [symbol] | Threonine | Thr | T |
| [symbol] | Cystine | Cys | C |
| [symbol] | Methionine | Met | M |
| [symbol] | Asparagine | Asn | N |
| [symbol] | Glutarnine | Gln | Q |
| [symbol] | Asparticacid | Asp | D |
| [symbol] | Glutamicacid | Glu | E |
| [symbol] | Lysine | Lys | K |
| [symbol] | Arginine | Arg | R |
| [symbol] | Histidine | His | H |

In a possible implementation method, the trained neural network model is obtained by training according to a method which comprises the steps of: carrying out clustering and characteristic extraction on the first database sequentially according to the classification of the antigens, the homology of the amino acid residues in the antigen epitopes, and the positions of the antigen recognition areas to obtain multi-dimensional vectors for predicting the antibody genes; and taking the multi-dimensional vector as the input of the temporal convolutional neural network, and stopping training until the error is lower than the threshold and tends to be stable to obtain the trained neural network model.

Schematically, the classification of the antigen epitopes and the corresponding recognition regions refers to the table 2:

TABLE 2
Classification and conventional properties of the antigen epitopes

| Properties | T cell epitope | B cell epitope |
|---|---|---|
| Epitope molecule | TCR | BCR |
| MHC molecule | Required | Not required |
| Epitope property | mainly linear polypeptide | natural polypeptide, |
| | | polysaccharide, |
| | | lipopolysaccharide and |
| | | organic compounds |
| Epitope size | 8-12 amino acids CDS-TC) | 5-15 amino acid |
| | 12-17 amino acids (CD4- | monosaccharide or 5-7 |
| | TC) | nucleotide |
| Epitope type | Linear epitope | conformation, linear |
| | | epitope table |
| Epitope position | any part of antigen | Surface of antigen |
| | molecule | molecule |

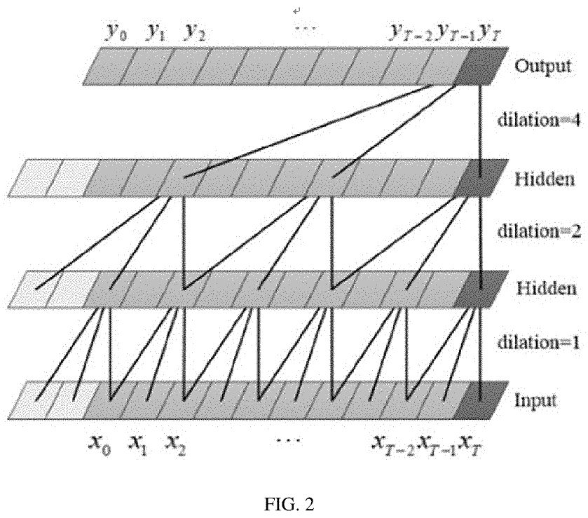

Referring to FIG. 2, the temporal convolutional network (TCN, also known as the temporal convolutional neural network) and comprises at least two convolutional hidden layers and at least one residual error module, wherein the output of at least one convolutional hidden layer is determined by a set number of latest label data, and the output of one convolutional hidden layer is determined by all label data. The above-mentioned label data refers to the labeled multidimensional vector for predicting the antibody genes. The xᵢ and yᵢ in the figure only represent input data and output data, and dilation represents the expansion coefficient.

Specifically, the residual error module uses the Zero-padding method to ensure that the dimensions of input and output data are consistent. The input received by the single residual error module is the output of the previous module (the first module receives source data input), and this data is used for calculating the result of a residual error block in one place, and for adding with the result of the residual error block through one-dimensional convolution in another place to serve as the output of this module. The part for calculating residual errors is firstly calculated by DilatedCasualConv; the historical information contained in the input data is calculated; in the present disclosure, the historical information is the preorder label information, then the historical information data is subjected to weight normalization (WeightNorm) and Non-linear transformation (ReLU) process, the results are controlled within a reasonable range, finally some results are randomly returned to zero through a random inactivation layer (Dropout), and the interdependence between modules is reduced. The time-related interest point data is extracted from part of data passing through a one-dimensional convolution layer (1×1Conv), and the residual error connection (+) is made with the historical information calculated by the residual error block, and the corrected data is obtained as the output of the current module. In this way, after multiple residual error modules are stacked and corrected, the output data TCN contains the required time interest point probability information for subsequent calculations. It can be understood that the label data in the above-mentioned time-domain convolutional neural network refers to the labeled multi-dimensional vector for predicting the antibody genes.

In a possible implementation method of the present disclosure, the screening out antibody sequences having different activities, stability and specificity to the antigens in the coding gene sequence set X according to molecular docking, molecular dynamics and the existing gene sequence database Y so as to establish a secondary antibody library comprises the steps of:

matching the coding gene sequence set X with the existing gene sequence database Y, calculating the similarity Sᵢ between coding gene sequences xᵢ and existing gene sequences yᵢ and arranging yᵢ, in descending order of similarity; and

taking the top 10 gene sequences of similarity as a candidate antibody sequence set, and establishing the secondary antibody library according to the activities, stability, and specificity of expression products of the candidate antibody sequence set.

Further, if the maximum value of the similarity Sᵢ of the candidate antibody sequences is lower than the threshold, the expression products of the candidate antibody sequences are subjected to molecular dynamics simulation or molecular docking with the antibodies in a simulated environment, and the activities, stability and specificity of the expression products are evaluated by a scoring function to establish the secondary antibody library.

It should be noted that in the calculation of the above similarity, distance formulas such as Mahalanobis distance and Euclidean distance can be used for measurement; and usually, the editing distance measurement comprises different editing operations. For example, the Damerau-Levenshtein distance allows the insertion, deletion, replacement and exchange of two adjacent characters; the longest common subsequence only allows insertion and deletion operations; the Hamming distance only allows replacement operation, and therefore, the Hamming distance is only suitable for two characters strings with the equal length. Preferably, the Damerau-Levinstein distance is used in the present disclosure.

Exemplarily, the specific steps of molecular docking comprise obtaining the structures of the proteins of the expression products through a PDB (protein data bank, a file format for three-dimensional structure information of the proteins). Currently, there are three crystal structures from different sources in the PDB, namely the yeast source, the human source, and the mouse source. For covalent molecular docking, polar hydrogen atoms and electric charges need to be added to a receptor protein file, and an appropriate docking area is selected according to the hydrophobic area of the protein surface; after the above steps are completed, the structures are saved as files in pdbqt format, and generate various coordinate files and map files of the receptor. For small-molecule ligands, the obtained structures are transformed into 3D structures with ChemDraw software, and ligand molecules are preprocessed by Racoon software to obtain the pdbqt files required for molecular docking. Then, the scoring function of the docking tool is used for evaluating the molecular structures of the antibodies, and the semi-empirical free energy calculation method of AutoDock software (molecular docking simulation software) is adopted, and has the docking accuracy higher than a Lamarck genetic algorithm of AutoDock software.

Exemplarily, on the premise that the expression products of the antibody sequence are the proteins, the specific steps of molecular dynamics simulation are as follows:

(1) applying a force field to the proteins by using Amber99sb Force Field (Hornak et al., Proteins 65, 712-725, 2006);

(2) pretreating small molecule compounds (adding the hydrogen atoms and the electric charges) through UCSF Chimera software, and generating force field parameter files by GAFF (general AMBER force field) and acpype in ANTECHAMBER;

(3) then putting a composite structure into a box, wherein the box is an octahedron that shows containing of water and has a boundary, and in order to make the whole system electrically neutral, adding an appropriate amount of sodium ions and chloride ions to the box so as to achieve equilibrium, wherein the minimum distance between the boundary of the box and each solute molecule is 10 angstroms;

(4) in order to achieve the best dynamic simulation and reduce the bad contact between solute and a solvent in the system, minimizing the energy of the system;

(5) performing temperature (nvt) balance and pressure (npt) balance on the system, performing molecular dynamic balance after making the system temperature rise from OK to 300K, and then performing dynamics balance of constant pressure and constant temperature on the system at a constant pressure of 1 atm and a temperature of 300K Kinetic balance; and

(6) performing molecular dynamics simulation, setting the cutoff value of non-bonded van der Waals interaction and electrostatic interaction as 10 angstrom, limiting the expansion and contraction of hydrogen-containing atomic bonds by adopting an LINCS algorithm, and calculating long-range electrostatic interaction by adopting a Particle-MeshEwald (PME) method.

The binding energy between the above composite systems is calculated by using a molecular mechanics/Poisson-Boltzmann surface area method (MM/PBSA). The formula used in this method is as follows: Δ G_binding=Δ E_MM+Δ G_solv−T Δ S_MM, Δ E_MM=Δ E_int+Δ E_vdW+Δ E_ele, Δ G_solv=Δ G_PB+Δ G_SA.

Referring to 3, the second aspect of the present disclosure provides an antibody library construction device based on deep learning 1, which comprises a construction module 11, a model training module 12, and a screening module 13. The construction module 11 is used for obtaining the corresponding relation among the antigen epitopes, the antigen recognition regions and the coding genes, and constructing the first database matching with the antigen epitopes, the antigen recognition regions and the coding genes; the model training module 12 is used for processing the antigen epitopes by using the trained neural network model so as to obtain the coding gene sequence set X of the antibodies to be predicted; and the screening module 13 is used for screening out the antibody sequences having different activities, stability and specificity to the antigens in the coding gene sequence set X according to molecular docking, molecular dynamics and the existing gene sequence database Y so as to establish the secondary antibody library.

Further, the screening module 13 comprises a calculating module 131, a first screening module 132, and a second screening module 133. The calculating module 131 is used for matching the coding gene sequence set X with the existing gene sequence database Y and calculating the similarity Sᵢ between the coding gene sequences xi and the existing gene sequences yᵢ; the first screening module 132 is used for arranging yᵢ in descending order of similarity, taking the top 10 gene sequences of similarity as the candidate antibody sequence set, and establishing the secondary antibody library according to the activities, stability, and specificity of the expression products of the candidate antibody sequence set; and the second screening module 133 is used for molecular dynamics simulation or molecular docking of the expression products of the candidate antibody sequences and the antibodies in a simulated environment in a simulated environment, and evaluating the activities, stability and specificity of the expression products by the scoring function to establish the secondary antibody library.

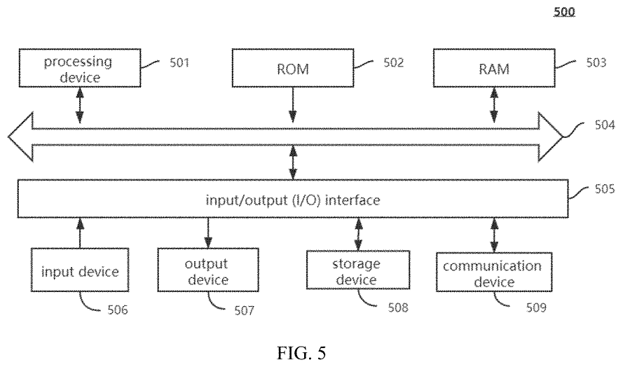

Referring to FIG. 5, electronic device 500 may comprise a processing device (such as a central processing unit and a graphics processor) 501, can execute various appropriate actions and processing according to programs stored in a read-only memory (ROM) 502 or programs which are loaded into a random access memory (RAM) 503 from a storage device 508. Various programs and data required for the operation of the electronic equipment 500 are also stored in the RAM 503. The processing device 501, the ROM 502, and the RAM 503 are connected to each other through a bus 504. An input/output (I/O) interface 505 is also connected to the bus 504.

Generally, the following devices can be connected to the I/O interface 505: including input devices 506 such as a touch screen, a touch tablet, a keyboard, a mouse, a camera, a microphone, an accelerometer and a gyroscope; including output devices 507 such as a liquid crystal display (LCD), a loudspeaker, and a vibrator; including a storage device 508 such as a hard disk; and a communication device 509. The communication device 509 may allow the electronic equipment 500 to perform wireless or wired communication with other equipment to exchange data. Although FIG. 5 shows the electronic equipment 500 provided with various devices, it should be understood that the implementation or arrangement of the illustrated devices is not required. More or fewer devices may be alternatively implemented or provided. Each block shown in FIG. 5 can represent one device or multiple devices as needed.

In particular, according to the disclosed embodiments of the present disclosure, the process described above with reference to the flowchart can be implemented as a computer software program. For example, the embodiment of the present disclosure comprises a computer program product, which comprises a computer program loaded on a computer-readable medium; and the computer program comprises program codes for executing the method shown in the flowchart. In such an embodiment, the computer program may be downloaded and installed from the network through the communication device 509, or installed from the storage device 508, or installed from the ROM 502. When the computer program is executed by the processing device 501, the above-mentioned functions defined in the method of the embodiment of the present disclosure are executed. It should be noted that the computer-readable media described in the embodiments of the present disclosure may be computer-readable signal media or computer-readable storage media, or any combination of the computer-readable signal media or the computer-readable storage media. The computer-readable storage media may be, for example, but not limited to, electrical, magnetic, optical, electromagnetic, infrared, or semiconductor systems, devices, or component, or a combination of any of the above. More specific examples of computer-readable storage media may comprise, but are not limited to: electrical connections with one or more wires, portable computer magnetic disks, hard disks, random access memories (RAM), read-only memories (ROM), erasable programmable read-only memories (EPROM or flash memory), optical fibers, portable compact magnetic disk read-only memories (CD-ROM), optical storage devices, magnetic storage devices, or any suitable combination of the above. In the embodiments of the present disclosure, the computer-readable storage medium may be any tangible medium that contains or stores the program, and the program may be used by or in combination with an instruction execution system, an apparatus, or a device. In the embodiments of the present disclosure, the computer-readable signal medium may comprise data signals propagated in a baseband or as a part of carrier waves, and computer-readable program codes are carried in the computer-readable signal medium. This propagated data signals can be in multiple forms, and include, but are not limited to electromagnetic signals, optical signals, or any suitable combination of the electromagnetic signals and the optical signals. The computer-readable signal media may also be any computer-readable media other than the computer-readable storage media the computer-readable signal media may send, propagate, or transmit the programs for use by or in combination with the instruction execution system, apparatus, or device. The program code contained in the computer-readable medium can be transmitted by any suitable medium, and includes, but are not limited to: wires, optical cables, RF (Radio Frequency), etc., or any suitable combination of the above.

The above-mentioned computer-readable medium may be included in the above-mentioned electronic equipment, or may exist alone without being assembled into the electronic equipment. The aforementioned computer-readable medium carries one or more computer programs, and when the aforementioned one or more programs are executed by the electronic equipment, the electronic equipment:

can write the computer program code for performing the operations of the embodiments of the present disclosure in one or more programming languages or a combination thereof, wherein the programming languages comprise object-oriented programming languages-such as Java, Smalltalk, C++, Python, and also comprise conventional procedural programming languages-such as “C” language or similar programming languages. The program code can be executed entirely on the user's computer, partly on the user's computer, executed as an independent software package, partly on the user's computer and partly executed on a remote computer, or entirely executed on the remote computer or server. In the case of involving a remote computer, the remote computer can be connected to the user's computer through any kind of network, including a local area network (LAN) or a wide area network (WAN), or can be connected to an external computer (for example, by using an Internet service provider through the Internet).

The flowcharts and block diagrams in the accompanying drawings illustrate the possible realized architecture, functions, and operations of the system, method, and computer program product according to various embodiments of the present disclosure. In this regard, each block in the flowcharts or block diagrams may represent a module, a program segment, or part of code, and the module, program segment, or part of code contains one or more executable instructions for realizing a specified logic function. It should also be noted that in some alternative implementations, the functions marked in the block may also occur in a different order from the order marked in the drawings. For example, two blocks shown in succession can actually be executed substantially in parallel, and they can sometimes be executed in the reverse order, depending on the functions involved. It should also be noted that each block in the block diagram and/or flowchart, and the combination of the blocks in the block diagram and/or flowchart, can be implemented by a dedicated hardware-based system that performs the specified functions or operations or can be realized by a combination of dedicated hardware and computer instructions.

The above are only the preferred embodiments of the present disclosure and are not intended to limit the present disclosure. Any modification, equivalent replacement, improvement, etc. made within the spirit and principle of the present disclosure shall be included in the protection scope of the present disclosure.

What is claimed is:

1. An antibody library construction method based on deep learning, comprising the steps of:
obtaining a corresponding relation among antigen epitopes, antigen recognition regions and coding genes, and constructing a first database matching with the antigen epitopes, the antigen recognition regions and the coding genes;
processing the antigen epitopes by using a trained neural network model so as to obtain the coding gene sequence set X of antibodies to be predicted, wherein the trained neural network model is obtained by training according to the method which comprises the steps of: carrying out clustering and characteristic extraction on the first database sequentially according to a classification of antigens, a homology of amino acid residues in the antigen epitopes, and positions of the antigen recognition areas to obtain a multi-dimensional vector for predicting antibody genes; and taking the multi-dimensional vector as the input of a temporal convolutional neural network, and stopping training until the error is lower than the threshold and tends to be stable to obtain the trained neural network model; and
screening out antibody sequences having different activities, stability and specificity to the antigens in the coding gene sequence set X according to molecular docking, molecular dynamics and an existing gene sequence database Y so as to establish a secondary antibody library.

2. The antibody library construction method based on deep learning of claim 1, wherein the temporal convolutional neural network comprises at least two convolutional hidden layers and at least one residual error module, the output of at least one convolutional hidden layer is determined by a set number of latest label data, and the output of one convolutional hidden layer is determined by all label data.

3. The antibody library construction method based on deep learning of claim 2, wherein the residual error module uses a Zero-padding method to ensure that dimensions of input data and output data are consistent.

4. The antibody library construction method based on deep learning of claim 1, wherein the screening out antibody sequences with different activities, stability and specificity to the antigens in the coding gene sequence set X according to molecular docking, molecular dynamics and the existing gene sequence database Y so as to establish the secondary antibody library comprises the steps of:
matching the coding gene sequence set X with the existing gene sequence database Y, calculating the similarity Sᵢ between coding gene sequences xᵢ and existing gene sequences yᵢ, and arranging yᵢ in descending order of similarity;
taking the top 10 gene sequences of similarity as a candidate antibody sequence set, and establishing the secondary antibody library according to the activities, stability, and specificity of expression products of the candidate antibody sequence set.

5. The antibody library construction method based on deep learning of claim 4, wherein if the maximum value of the similarity Sᵢ of the candidate antibody sequences is lower than the threshold, the expression products of the candidate antibody sequences are subjected to molecular dynamics simulation or molecular docking with the antibodies in a simulated environment, and the activities, stability and specificity of the expression products are evaluated by a scoring function to establish the secondary antibody library.

6. An antibody library construction device based on deep learning, comprising a construction module, a model training module, and a screening module, wherein
the construction module is used for obtaining the corresponding relation among the antigen epitopes, the antigen recognition regions and the coding genes, and constructing the first database matching with the antigen epitopes, the antigen recognition regions and the coding genes;
the model training module is used for processing the antigen epitopes by using the trained neural network model so as to obtain the coding gene sequence set X of the antibodies to be predicted; the trained neural network model is obtained by training according to the a method which comprises the steps of: carrying out clustering and characteristic extraction on the first database sequentially according to the classification of the antigens, the homology of the amino acid residues in the antigen epitopes, and the positions of the antigen recognition areas to obtain the multi-dimensional vector for predicting the antibody genes; and taking the multi-dimensional vector as the input of the temporal convolutional neural network, and stopping training until the error is lower than the threshold and tends to be stable to obtain the trained neural network model; and
the screening module is used for screening out the antibody sequences having different activities, stability and specificity to the antigens in the coding gene sequence set X according to molecular docking, molecular dynamics and the existing gene sequence database Y so as to establish the secondary antibody library.

7. The antibody library construction method based on deep learning of claim 6, wherein the screening module comprises a calculating module, a first screening module and a second screening module, wherein
the calculating module is used for matching the coding gene sequence set X with the existing gene sequence database Y and calculating the similarity Sᵢ between the coding gene sequences xi and the existing gene sequences yᵢ;
the first screening module is used for arranging yᵢ in descending order of similarity, taking the top 10 gene sequences of similarity as the candidate antibody sequence set, and establishing the secondary antibody library according to the activities, stability, and specificity of the expression products of the candidate antibody sequence set; and
the second screening module is used for molecular dynamics simulation or molecular docking of the expression products of the candidate antibody sequences and the antibodies in a simulated environment, and evaluating the activities, stability and specificity of the expression products by the scoring function to establish the secondary antibody library.
